(19) United States
(12) Patent Application Publication
(10) Pub. No.: US 2019/0221318 A1
(43) Pub. Date: Jul. 18, 2019

(54) SYSTEMS, METHODS AND TECHNIQUES FOR SOCIAL MEDIA MULTI-MODAL HEALTH TECHNOLOGY PLATFORM

(71) Applicant: JOY HEALTH SYSTEMS, LLC, Baltimore, MD (US)

(72) Inventor: Tonamy Joy PATERAKIS, Baltimore, MD (US)

(21) Appl. No.: 16/245,539

(22) Filed: Jan. 11, 2019

Related U.S. Application Data
(60) Provisional application No. 62/616,500, filed on Jan. 12, 2018.

(51) Int. Cl. G16H 80/00; G06Q 50/00; G06F 3/0482; G06F 3/0488; G06F 3/0484

(52) U.S. Cl. CPC G16H 80/00; G06Q 50/01; G06F 3/0482; G06F 2203/04808; G06F 3/04845; G06F 2203/04806; G06F 3/04883

(57) ABSTRACT

Systems, methods and techniques for a social media multi-modal health platform are disclosed. The platform includes a Profile Module relating to profile information of a user of the platform, an Identity Module relating to various categories or determinants of health, a Social Module relating to posts posted by the user of the platform, a Planet Module showing user posted content in a pixilated planet, a Market Module relating to advertisements and products that are harmonious to one or more of the categories of health on the Identity Module, a Why Module relating to one or more of the determinants or categories of health in the Identity Module, and an Entry Module enabling the user of the platform to enter information.

### CROSS REFERENCE TO RELATED APPLICATIONS

This application is a non-provisional of U.S. Application No. 62/616,500, filed Jan. 12, 2018, the entire contents of which are incorporated herein by reference.

### FIELD OF THE DISCLOSURE

Example embodiments disclosed herein relate generally to social media multi-modal health technology platforms, and more particularly to systems, methods and techniques for social media multi-modal health technology platforms integrating social media communication with multi-modalities of health.

### BACKGROUND AND SUMMARY

Health application availability and use has been on the rise recently, and there are mobile applications for smart phones that aid in increasing health & fitness in some form. Examples include apps for tracking running, sleep cycles, menstruation cycles, blood pressure, blood glucose, footstep count, calories, nutrition, along with offering yoga classes, fitness workouts, counseling, meditation, food education, and many others. Health apps are heavily used by smartphone owners to aid in increasing their health in a more accessible and/or affordable way, and variability is wide for scope of health app availability.

There are some apps that also aid in connecting groups to make health more social, including syncing workout groups, or running groups, for example. Many of the health apps, however, only focus on one specific category of health. Apple's Health app via HealthKit, is the only other app on the market that has integrated more categories of health (including exercise, nutrition, sleep, and more medical data), though void of social media structure. Furthermore, SnapChat, also incorporates swiping, into their directional navigation throughout the platform, but then again that is also seen in other mobile applications though perhaps less emphasized, but still present. However, SnapChat is void of a thematic focus of integrated health in its design. Those are existing applications that came up within the landscape territory for comparison. Additionally, there may also be general crossover, by nature of this application invention being under the umbrella of a social media platform, with Facebook and/or Twitter features (i.e. including the function to direct message, or for posting options via text, emojis, photos, videos, and life events on the running social page, along with the functionality behind Facebook's “Friends” and “Add Friends,” and similarly Twitter's “Followers” and “Add Followers”), thus mentioned.

Example embodiments are directed to a new and improved, along with redesigned, health platform that not only offers the main modalities (categories) of health in its structure and function, but also does it by integrating social media communication structurally and functionally into the platform.

In certain example embodiments, the health application includes categories of health and social medial communication technology. Example embodiments of the invention function to connect multi-modal health categories (aka modalities) with social media communication technology in a mobile application format, along with secured inclusion for its related website design.

In certain example embodiments, an improved, multi-modal health and integrated social application (e.g., a mobile app) or website provides at least five main pages that will be described in further detail, and other non-main pages linked in from there.

According to one aspect of the present technology, there is provided a social media multi-modal health technology platform to help motivate people towards health balance in ways that are self-driven and socially-driven. Concretely stated, the platform is being built to increase health status & health goal awareness, motivate, and be social, for its users, along with simultaneously being profitable in the platform's business model. The reason for building this platform is founded on the importance of seeing where one currently is, in juxtaposition with big-picture goal(s) for where one wants to be, along with being strongly motivational to cheer others (i.e. family/friends, aka one's “squad”) along their health journey towards their health goals. The platform will also potentially add a feature that connects celebrities to send motivational messages to the respective app users, for increased motivation. Additionally, the platform will address the whys, whats, and hows of behavior change, and will be structured to facilitate more conscientious and self-directed behavior change.

One example of the present technology is directed to a multi-modal health and social media system comprising a processing system including at least one processor. The processing system is configured to generate a data entry user interface enabling a user to enter information related to at least one modality of health. The entered information includes a current value of the at least one modality of health accessed by the user and a desired value of the at least one modality of health from the user. The processing system is further configured to convert the entered information related to the at least one modality of health into converted information related to the at least one modality of health, represent the converted information related to the at least one modality of health on a modality review user interface, generate a post entry user interface enabling the user to submit one or more posts to a social media platform, and display the posts submitted by the user as corresponding data entries on a planet respectively.

In an example embodiment, the at least one modality of health comprises body, mind, soul, air, water, nutrition, exercise, community, sleep, shelter and/or love.

In another example embodiment, the converted information includes information generated based on the current value of the at least one modality of health, and/or the desired value of the at least one modality of health. In an embodiment, the converted information includes an average current value of the at least one modality of health averaged from the current value and other current values of the at least one modality of health previously entered by the user during a time period, and an average desired value of the at least one modality of health averaged from the desired value and other desired values of the at least one modality of health previously entered by the user during the time period.

In yet another example embodiment, the submitted one or more posts are added to the planet as pixilated formations. For example, the submitted one or more posts comprises textual information, image(s), audio(s), and/or video(s).

In some example embodiments, the processing system is further configured to detect a user input corresponding to a directional swipe, and, upon detecting the user input, cause the planet to rotate towards a direction indicated by the directional swipe.

In other example embodiments, the processing system is further configured to cause the planet to zoom in or zoom out in accordance with a user input.

In an example embodiment, the processing system is further configured to display details of a post upon detecting a data entry corresponding to the post is selected by a user. In another embodiment, the processing system is further configured to generate a user interface enabling the user to edit the details of the post.

One example of the present technology is a non-transitory computer-readable storage medium having stored therein an information processing program for execution by a device comprising a processor operatively coupled to a display, the information processing program comprising instructions that, when executed by the processor, causing the device to perform operations including generating a data entry user interface enabling a user to enter information related to at least one modality of health. The entered information may include both a current value of the at least one modality of health accessed by the user and a desired value of the at least one modality of health from the user. The information processing program may also comprise instructions that, when executed by the processor, causing the device to perform operations including converting the entered information related to the at least one modality of health into converted information related to the at least one modality of health, representing the converted information related to the at least one modality of health on a modality review user interface, generating a post entry user interface enabling the user to submit one or more posts to a social media platform, and displaying the posts submitted by the user as corresponding data entries on a planet respectively.

In an example, the at least one modality of health comprises body, mind, soul, air, water, nutrition, exercise, community, sleep, shelter and/or love.

In another example, converting the entered information related to the at least one modality of health into the converted information related to the at least one modality of health comprises generating an average current value of the at least one modality of health by averaging the current value and other current values of the at least one modality of health previously entered by the user during a time period, and generating an average desired value of the at least one modality of health by averaging the desired value and other desired values of the at least one modality of health previously entered by the user during the time period.

In yet another example, the submitted one or more posts are added to the planet as pixilated formations. In some examples, the information processing program further comprises instructions that, when executed by the processor, causing the device to perform operations including detecting a user input corresponding to a directional swipe, and, upon detecting the user input, causing the planet to rotate towards a direction indicated by the directional swipe. In other examples, the information processing program further comprises instructions that, when executed by the processor, causing the device to cause the planet to zoom in or zoom out in accordance with a user input.

Some examples are directed to a method for managing and sharing multi-modal health information, comprising generating a data entry user interface enabling a user to enter information related to at least one modality of health. For example, the entered information includes a current value of the at least one modality of health accessed by the user and a desired value of the at least one modality of health from the user. The method may further comprise converting the entered information related to the at least one modality of health into converted information related to the at least one modality of health, representing the converted information related to the at least one modality of health on a modality review user interface, generating a post entry user interface enabling the user to submit one or more posts to a social media platform, and displaying the posts submitted by the user as corresponding data entries on a planet respectively.

In an example, the submitted one or more posts are added to the planet as pixilated formations.

In another example, the method may further comprise detecting a user input corresponding to a directional swipe, and, upon detecting the user input, causing the planet to rotate towards a direction indicated by the directional swipe.

### BRIEF DESCRIPTION OF THE DRAWINGS

The accompanying drawings facilitate an understanding of the various preferred embodiments of this invention. In such drawings:

FIG. 1 shows a non-limiting, exemplary block diagram for an example social media multi-modal health technology platform;

FIG. 2 shows a non-limiting, exemplary block diagram of an example mobile communication device;

FIG. 3 shows a non-limiting, exemplary user interface provided by an example social media multi-modal health technology platform—a Profile Page;

FIG. 4 shows a non-limiting, exemplary user interface provided by an example social media multi-modal health technology platform—an Identity Page;

FIG. 5 shows a non-limiting, exemplary user interface provided by an example social media multi-modal health technology platform—a Social Page;

FIG. 6A shows a non-limiting, exemplary user interface provided by an example social media multi-modal health technology platform—a Planet Page;

FIG. 6B shows a non-limiting, exemplary user interface provided by an example social media multi-modal health technology platform—a Planet Page;

FIG. 7 shows a non-limiting, exemplary user interface provided by an example social media multi-modal health technology platform—a Market Page;

FIG. 7A shows a non-limiting, exemplary user interface provided by an example social media multi-modal health technology platform—a Market Page;

FIG. 7B shows a non-limiting, exemplary user interface provided by an example social media multi-modal health technology platform—a Market Page;

FIG. 7C shows a non-limiting, exemplary user interface provided by an example social media multi-modal health technology platform—a Market Page;

FIG. 7D shows a non-limiting, exemplary user interface provided by an example social media multi-modal health technology platform—a Market Page;

FIG. 7E shows a non-limiting, exemplary user interface provided by an example social media multi-modal health technology platform—a Market Page;

FIG. 8A shows a non-limiting, exemplary user interface provided by an example social media multi-modal health technology platform—a Why Page;

FIG. 8B shows a non-limiting, exemplary user interface provided by an example social media multi-modal health technology platform—a Why Page;

FIG. 8C shows a non-limiting, exemplary user interfaces provided by an example social media multi-modal health technology platform—a Why Page;

FIG. 8D shows a non-limiting, exemplary user interfaces provided by an example social media multi-modal health technology platform—a Why Page;

FIG. 9A shows a non-limiting, exemplary user interface provided by an example social media multi-modal health technology platform—an Entry Page;

FIG. 9B shows a non-limiting, exemplary user interface provided by an example social media multi-modal health technology platform—an Entry Page; and

FIG. 10 shows a non-limiting, exemplary user interface provided by an example social media multi-modal health technology platform—an entry average of entry pages.

### DETAILED DESCRIPTION OF EXEMPLARY EMBODIMENTS

In accordance with certain exemplary embodiments, certain systems, device, processes and methods are disclosed for social media multi-modal health technology platforms, more particularly, certain exemplary embodiments relating to social media multi-modal health technology platform integrating both social media with multi-modalities of health are described herein. In the following description, for purpose of explanation, numerous specific details are set forth to provide a thorough understanding of the exemplary embodiments. It will be evident, however, to a person skilled in the art that the exemplary embodiments may be practiced without these specific details. To note, details that are outside of the claim are non-binding, and can be modified stylistically at will for the purpose of the overall invention's chosen design creation.

Example embodiments of the present technology provide social media multi-modal health technology platform configured to manage and share information relevant to human health, and enable users to record, monitor and improve their health. The range of personal, social, economic, and environmental factors that influence health status are determinants or modalities of health, for example, body, mind, soul, air, water, nutrition, exercise, community, sleep, shelter and/or love, etc. The determinants or modalities of health may fall under different categories, such as individual behaviors, social factors, biology and genetics, health services, policymaking, etc.

One example of the present technology disclosed herein allows digital-visual representation for numerical data entry averages of where one would like to be (e.g., health vision-orientation, namely digital-visual representation of health vision) for a particular health modality or determinant. Certain example embodiments provide the ability to digitally and visually represent health vision, which is different than health goal(s). Goals are short term, require action for attainability, are limited, realistic, time-specific, and produce results. Vision is long term, does not require action to be attainable, is unlimited, imaginative, non-time specific, and unattainable when unpaired with goals. The important and critical difference between a goal and a vision is common understanding for business structures and developments, along with behavioral medical and behavioral counseling techniques for change. However, health vision has yet to have been represented in digital-visual format. The present technology therefore provides a digital-visual health system that represents health vision digitally and visually via averages and/or digital-visual representations. For example, multi-media posts and data entries submitted by users will be converted into digital-visual representations, and displayed to relevant users.

As well, another example of the present technology provides the ability to digitally represent health vision integrated with social media digital platform format(s). This digital and data-driven socialization of digital and data-driven representation for health vision actualization through digital, data-driven, and digital-visual media means, in which digital and data-driven socialization is defined as the digital and data-driven ability and activity of interfacing and interacting socially with other users, i.e. via a social page and notifications, and related social media technology bidirectional, group, and public communications, i.e. postings, messages, and chat groups. For example, an example embodiment of the present technology enables users to post in ways that allow users to motivate other users. It socially motivates, educates, and increases users to a health marketplace all in one space and essentially forms an online social media platform for health determinants and motivationally supports users inter-socially to reach their desired ratings for determinants of health of where they would like to be, alongside forming social community space to post content, socially interact, self-assess determinants of health ratings, and purchase and interact with advertised health goods.

Digital health vision actualization will be accomplished through self-rating user inputs of digital information converted into visual information via self-rating digital-visual systems and/or self-privatizing digital-visual and data-driven systems, in which the later term will be further described below, to assess and accomplish vision actualization for health modalities' and determinants' foundational digital-visual and data-driven means of user data representation. The self-rating digital-visual systems and/or self-privatizing digital-visual and data-driven systems are described more specifically below, i.e. through the identity page, planet page, and market page features that are all contributive towards increasing the platform's vision actualization for the user overall, thus termed cumulatively digital-visual health actualization.

An example of the present technology provides a system to digitally and visually represent health vision actualization through user privatized digital, data-driven, and digital-visual media means. Privatized information can be defined by the user abstaining from the release of information being accessible by another user's digital and data-driven interfacing and interacting means (whether it be single users, multiple users, or publicized access to users). This option for privatization of user information allows for an individual user to increase publicity or privacy of content, according to their own individual needs and digital preferences.

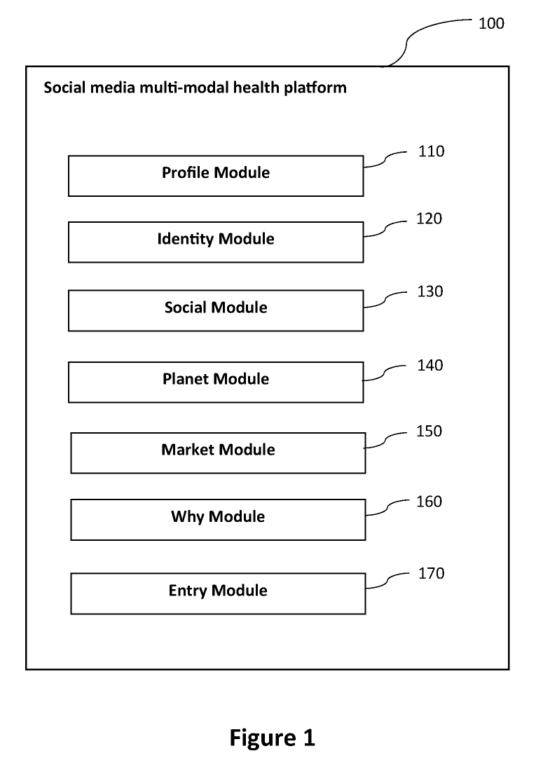

FIG. 1 shows a non-limiting, exemplary block diagram for an example social media multi-modal health technology platform 100. Some example embodiments may have different and/or other sub-modules than the ones described herein. Similarly, the functions can be distributed among the sub-modules in accordance with other embodiments in a different manner than is described herein.

The social media multi-modal health technology platform 100 includes modules supporting various functionalities of the platform, for example, a Profile Module 110, an Identity Module 120, a Social Module 130, a Planet Module 140, a Market Module 150, a Why Module 160 and an Entry Module 170. The Profile Module 110 relates to profile information of a user of the social media multi-modal health platform 100, for example, including settings of the user, and the user's friends, fans, and/or followers, etc. In an example embodiment, the friends are similar to “Friends” in Facebook, and the followers are similar to “Followers” in Twitter. The Identity Module 120 relates to various categories or determinants of health, for example, body, mind, soul, air, water, nutrition, exercise, community, sleep, shelter and/or love, etc. The Social Module 130 relates to posts posted by the user of the social media multi-modal health technology platform 100. The Planet Module 140 displays posts, such as pictures, images, and/or videos, etc., in the form of a pixilated planet, in which all of the posted content will be added to the planet as pixilated formations. The Market Module 150 relates to advertisements and products that are harmonious to one or more of the categories of health on the Identity Module 120. The Why Module 160 provides pages related to one or more of the determinants or categories of health in the Identity Module 120. The Entry Module 170 enables users of the social media multi-modal health technology platform 100 to enter information into the platform, such as rating himself/herself on a health scale of 1-10.

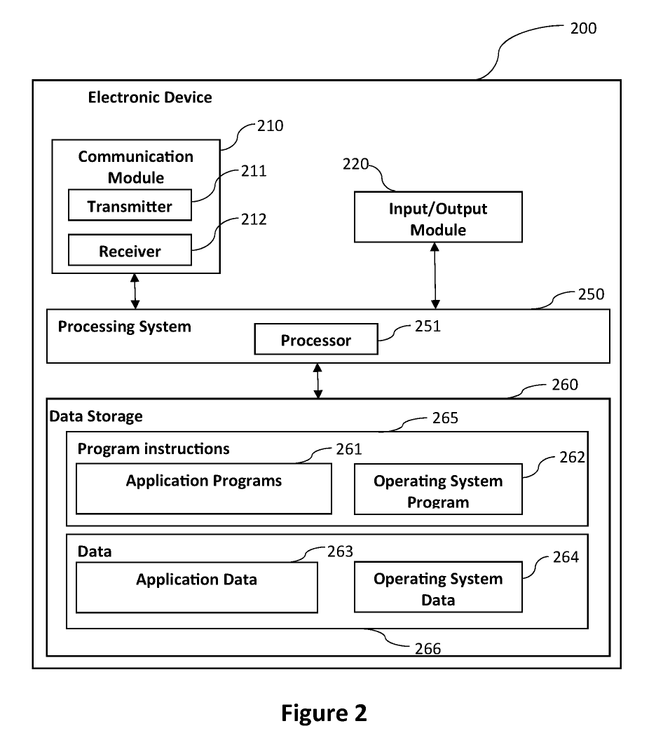

It is envisioned that the social media multi-modal health technology platform 100 is running on an electronic device. In certain example embodiments, the electronic device can be a mobile phone, a PDA, a laptop, or any other electronic devices equipped with one or more wireless and/or wired communication interfaces, though it will be appreciated that other properly configured devices may be used. FIG. 2 shows a non-limiting, exemplary block diagram of an example electronic device 200. In this exemplary embodiment, the electronic device 200 includes a communication module 210, an input/output module 220, a processing system 250 and a data storage 260, all of which may be communicatively linked together by a system bus, network, or other connection mechanisms.

The communication module 210 functions to allow the electronic device 200 to communicate with one or more of the other devices. The communication module 210 comprises a transmitter 211 configured to transmit data to other devices and a receiver 212 configured to receive data from other devices.

In certain example embodiments, the communication module 210 may comprise one or more communication interfaces supporting satellite communications, radio communications, telephone communications, cellular communications, internet communications, and/or the like. In other example embodiments, the communication module 210 may comprise a wireless transceiver with connected antenna, a wireless LAN module, a radio-frequency (RF), Infrared, or Bluetooth® transceiver, and/or a near field communication transceiver module. One or more of these communication components may collectively provide a communication mechanism by which the electronic device 200 can communicate with other devices, platform and/or networks.

The data storage 260 may comprise one or more volatile and/or non-volatile storage components, such as, a hard disk, a magnetic disk, an optical disk, read only memory (ROM) and/or random access memory (RAM), and may include removable and/or non-removable components. The date storage 260 may be integrated in whole or in part with the processing system 250.

The processing system 250 may comprise one or more processors 251, including one or more general purpose processors and/or one or more special purpose processors (i.e., DSPs, GPUs, FPs or ASICs). The processing system 250 may be capable of executing application program instructions 265 (e.g., compiled or non-compiled program and/or machine code) stored in data storage 260 to perform the various functions and processes described herein. The data storage 260 may include non-transitory computer-readable medium, having stored thereon program instructions that, if executed by the electronic device 200, cause the electronic device 200 to perform any of the processes or functions disclosed herein and/or illustrated by the accompanying drawings.

In certain example embodiments, the program instructions 265 may include an operating system program 262 and one or more application programs 261, such as program instructions for a social media multi-modal health technology platform installed on the electronic device 200. Further, the application programs 261 may communicate with the operating system program 262 via certain application programming interfaces.

During the execution of the program instructions 265, the processing system 250 may use data 266 including application data 263 and operating system data 264. In particular, the operating system data 264 may be accessed while the processing system 250 is executing the operating system program 262, and the application data 263 may be accessed while the processing system 250 is executing the application programs 261.

The input/output module 220 of the electronic device 200 may function to allow the electronic device 200 to interact with a human or non-human user, such as to receive inputs from a user and to provide outputs to the user. The input/output module 220 may include a touch-sensitive or presence-sensitive panel, keypad, keyboard, trackball, joystick, microphone, still camera and/or video camera, and the like. The input/output module 220 may also include one or more output components such as a display device, which may be combined with a touch-sensitive or presence-sensitive panel. Examples of the display device may include a cathode ray tube (CRT), a liquid crystal display (LCD), an electro luminescence (EL) display, a vacuum fluorescent display, a plasma display panel, a thin-film transistor (TFT) LCD, an organic light-emitting display device, an electrophoretic display, and the like. In an example embodiment, the input/output module 220 may display various user interfaces to enable a user to access services or functions provided by the electronic device 200. The input/output module 220 may also be configured to generate audible output, via a speaker, audio output port, audio output device, earphones, and/or other similar devices.

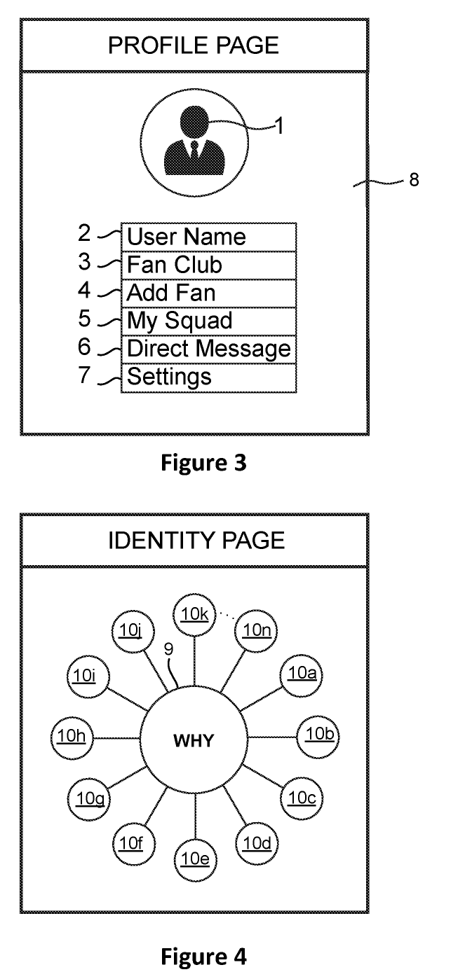

FIG. 3 shows a non-limiting, exemplary user interface provided by an example social media multi-modal health technology platform—a Profile Page. Example elements of the Profile Page are described herein. Item 1 may be the space where a profile picture would go. In an example embodiment, Item 1 is depicted as circular in shape. Item 2 may be a placeholder, for example, for the username. Item 3 may be a placeholder, for example, for the Fan Club (or “My Fans”). Item 4 may be a placeholder, for example, for “Add Fans” (or “Invite Fans”), which enables a user to add members to gain access to viewing the user's pages. In an example embodiment, the “Add Fans” may be similar to “Friends” or “Add Friends” in Facebook or “Followers” or “Add Followers” in Twitter. For example, pages of a user may only be viewed by those who are accepted to be the user “Fans.” Item 4 may also list namely those who the user has formally accepted to have access to socially motivate the user towards his/her health goals. In an example embodiment, clicking on “Add Fans” will take a user to a search page with a search bar to find more people on this application or website.

In certain example embodiments, a user may connect the social media multi-modal health technology platform with his/her contacts on his/her personal devices, such as, a phone, an iPad, etc., to automatically synchronize potential friends within the platform with his/her personal contacts from one or more of these devices. Item 5 may be a placeholder, for example, for “My Squad,” which is similar to “My Friends” in Facebook or “My Followers” in Twitter. In an example embodiment, My Squad lists who the user is following/motivating towards their health goals. In other example embodiments, alternate titles in place of “Squad” are open to be considered and modified, of which the name itself is not bound within the details of this disclosure.

Item 6 may be a placeholder, for example, for “Direct Messages” (hereinafter “DMsgs”), relating to messaging and communication. For example, DMsgs is for directing messages to and from (inbox and outbox, i.e. 2 tabs) individuals or groups within the health technology platform. In one embodiment, the platform may allow the DMsgs to be adjusted in Settings, for instance, to either receive messages only from Fans or the user's Squad, or to receive messages from the public. Item 7 may be a placeholder, for example, for the Settings, which enables a user to change and personalize preferences in the health technology platform. The Settings section/tab allows users to make all kinds of personalized adjustments within the platform. In another embodiment, the profile page also may have the option to allow for motivational messages received by the user's fan club, to be selected and displayed on the profile page in its extra space (see e.g., Item 8), for increased visible motivation on the user's profile page and around the user's profile picture.

One example of the present technology provides the ability for user data, specific to health modalities and determinants to be transferred into the numerical and visual representations. The health modalities and determinants are defined by topical bodies or integral components respectively for health foundations, e.g., nutrition, exercise, hydration, sleep, stress management, spirituality, etc. FIG. 4 shows a non-limiting, exemplary user interface provided by an example social media multi-modal health technology platform—an Identity Page. In this embodiment, the Identity page may display the main determinants or categories of health that will be focused on, for instance, Body, Mind, Soul, Air, Water, Nutrition, Exercise, Community, Sleep, Shelter, and/or Love, etc.

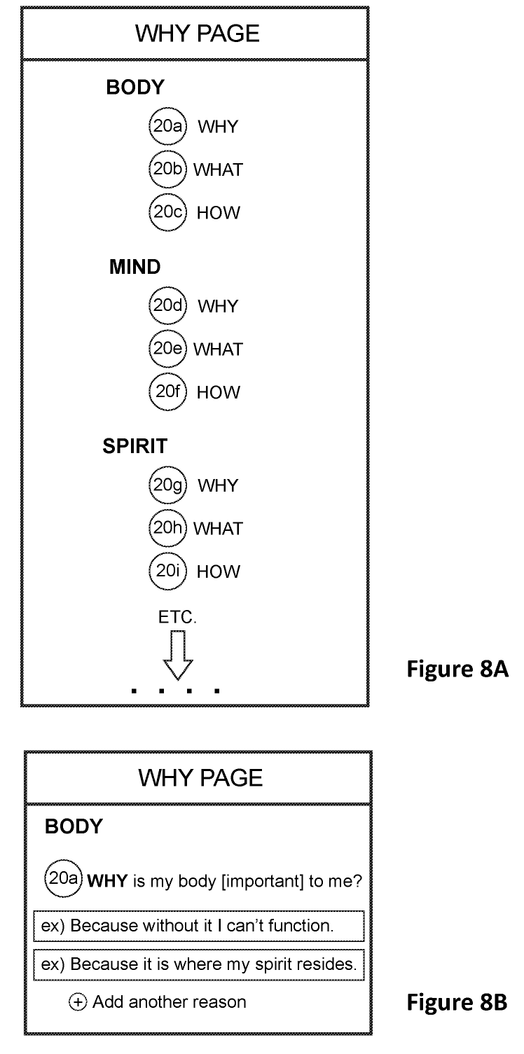

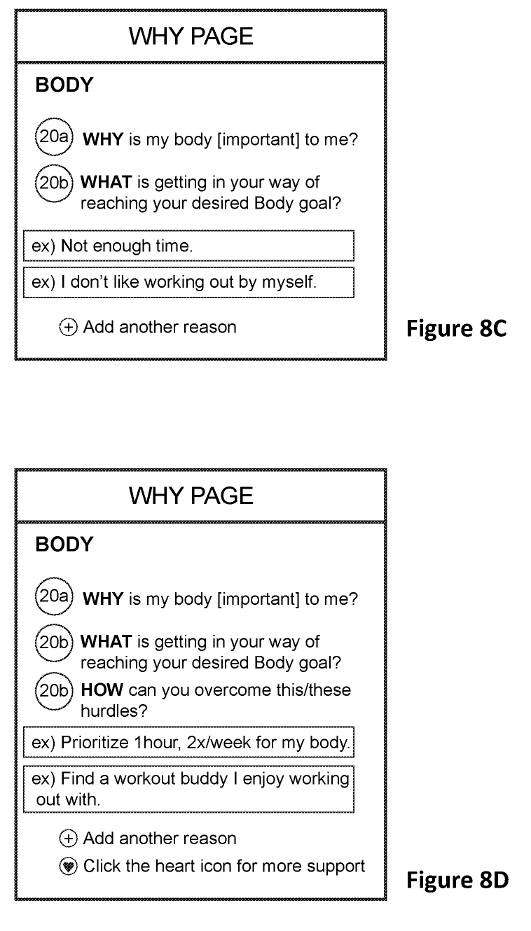

In an embodiment, at the center of these categories of health, Item 9 may list for example, “WHY”, which will take a user to a WHY Page, as shown in FIGS. 8A-8D, and which will be described in detail below in connection with FIGS. 8A-8D.

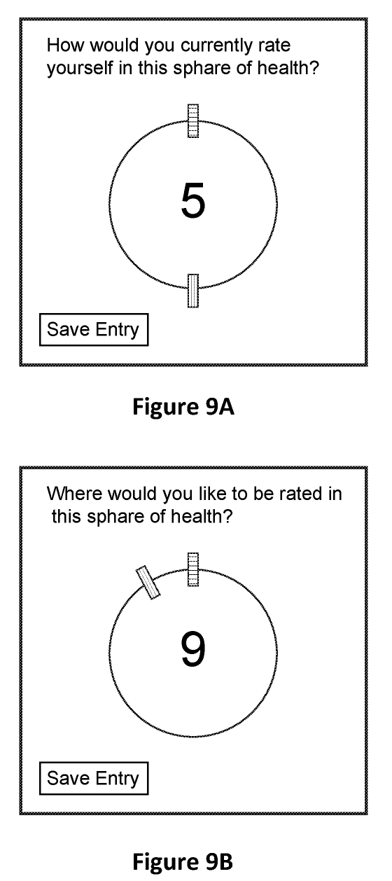

The Identity Page may also include a plurality of items, such as Items 10a, 10b, 10c, 10d, 10e, 10f, 10g, 10h, 10i, 10j, 10k, etc. to 10(n), where n is any number of added pages (or spheres of health). These items may correspond to various determinants of health (modalities or categories of health) respectively. For instance, item 10f may correspond to one of the determinants, modalities or categories—“BODY.” Pressing on item 10f will take a user to the Entry Pages for this determinant or modality of health—“BODY”, which allows the user to enter information related to the particular determinant or modality of health. Other example Entry pages are described in detail below in connection with FIGS. 9A-9B.

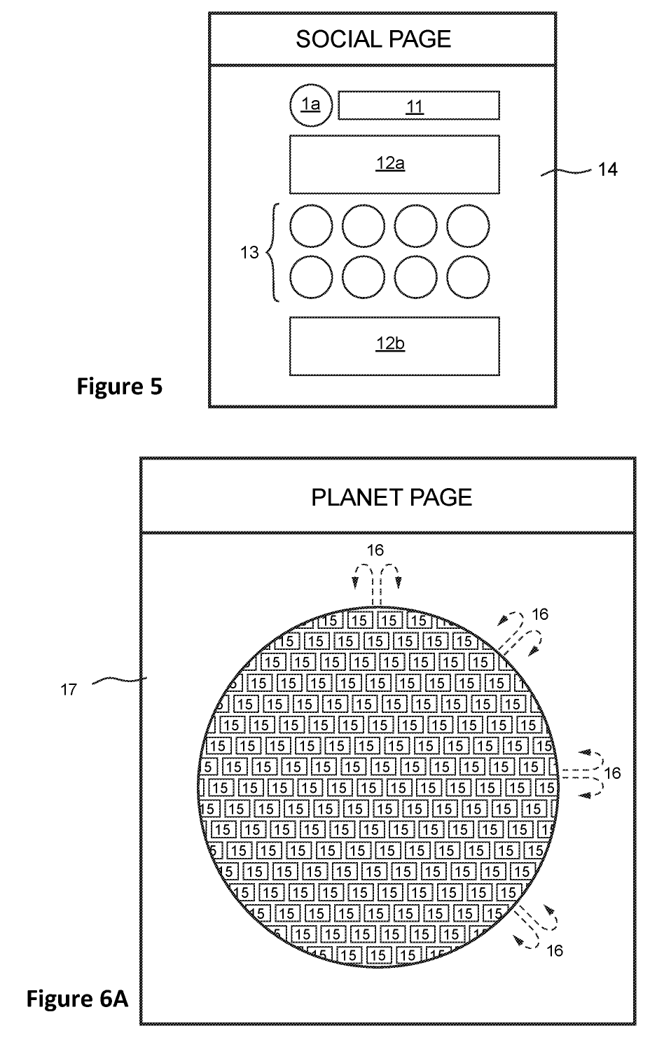

FIG. 5 shows a non-limiting, exemplary user interface provided by an example social media multi-modal health technology platform—a Social Page. In certain example embodiments, the Social Page is the main page for all personal posts made by the user in charge (I.D.'ed by the username and profile picture). Item la may be a placeholder for representing, for example, the chosen profile picture of the user that stands next to posts to-be made, or made by the user. Alternatively, a “Thumbnail” version of the profile picture of a user may be used for the picture representing the user's posts. Item 11 may be a placeholder, for example, for text, Emojis, Photos, Videos, and Life Events/Updates, within this social page's status entry for data subject matter (of course this is not a bound detail to the disclosure). Additionally, posts made have been considered to be made in text boxes, as depicted for example in Item(s) 12a and 12b, and pictures sent in spheres depicted for example in Item(s) 13, to foster retaining the spherical theme through the platform. Also, motivational messages have been proposed to be adjusted in settings to be displayed in some form to-be openly and freely determined, in the spaces marked by Item(s) 14, allowing for motivational messages to be integrated within the social page visual display.

In some embodiments, health vision is actualized through user privatized digital-visual and data-driven means, in which privatized information is defined by the user abstaining from releasing data and information to be accessible by another user's digital interfacing and interacting means, whether it be single users, multiple users, or publicized access to users.

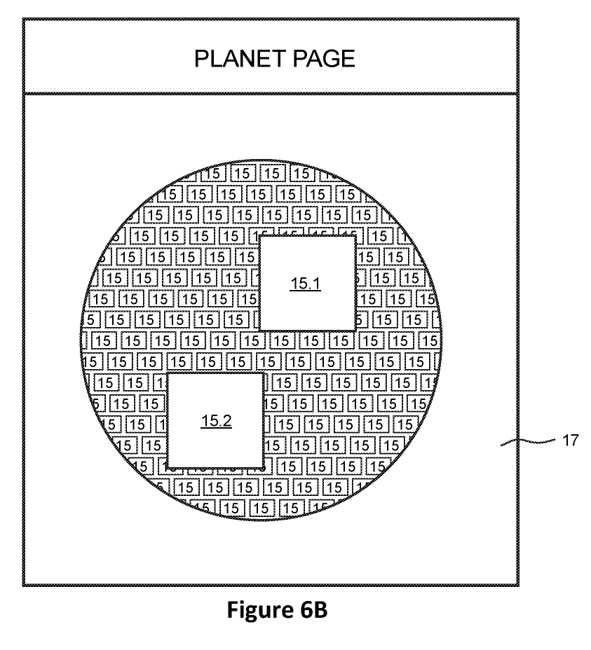

FIGS. 6A and 6B show non-limiting, exemplary user interfaces provided by an example social media multi-modal health technology platform—Planet Pages. Digital and data-driven visual media data information (e.g., images, videos, iconographs, etc.) can be posted and transferred from social pages to a planet page, in which the planet page can be edited as well, effectively allowing the user to create health vision actualization for themselves through this page as well, namely, FIG. 6B and bolstered within Items 15, 15.1, 15.2, and 17.

In an example embodiment, a user's social page posts (or selected content adjusted in settings) are depicted by Item(s) 15 on the Planet Page. The planet page will be in the form of a pixilated planet, in which all of the posted content (or selected content altered in Settings), as depicted Item(s) 15, will be added to the planet as pixilated formations. Items(s) 15 shown on the pixilated planet may comprise textual information, pictures, images, and/or videos, etc. Another example of the present technology disclosed herein provides a digital-visual and data-driven system to intake user data for health modalities or determinants and to convert (and transfer) it into digital-visual and data-driven media to create health vision (i.e. on a planet page), and thus contribute to digital-visual health actualization.

These pixilated formations will be of appropriate proportional size. The more social page posts or selected content in the planet, the smaller each box or sphere (design of this dynamic is not-bound by the disclosure, and thus remains open for reconstruction) for added pixilated content to the planet, and inversely, the less social page posts or selected content, the bigger the boundary aka taken up space, for each pixilated unit of content in the user's planet. This planet will be able to be adjusted in settings, to delete the content or edit it accordingly, as per user's preferences. In an embodiment, the planet will be able to be rotated manually via directional swiping as depicted, for example, by Item(s) 16, and zooming in and out, via appropriate manual screen movements that are pre-adjusted settings from user's phone (phone settings). This rotation dynamic can be respectively done by the user and by viewers of the user's Planet page. The background, depicted by Item(s) 17, may be adjusted via the settings section of the profile page discussed above.

The planet page will allow for manipulation and management of data (e.g., content and posted visual data items therein) via rotation of the total planet, expansion of the planet (e.g., zoom in), contraction of the planet (e.g., zoom out), selection and isolation of certain planet entries (e.g., as represented by Item(s) 15 in FIGS. 6A-6B and Item(s) 15.1-15.2 in FIG. 6B), along with the ability to bring data content into a larger, selected zoomed-in view to visualize particular planet entries (e.g., Item(s) 15.1-15.2 in FIG. 6B).

In some embodiments, the planet entries, as a side note, can be directly edited from the planet page. A user may click on one of the planet entries and then details of the planet entry will be displayed to the user on the planet or on another page. Further, the user may choose to edit the details of the entry or add additional information to the entry, e.g., textual information, image(s), visual content, and/or audial content.

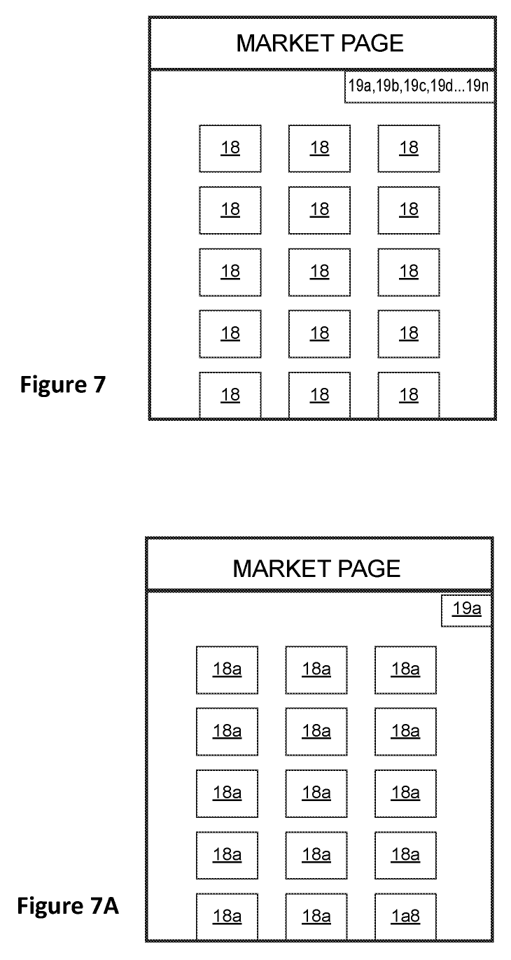

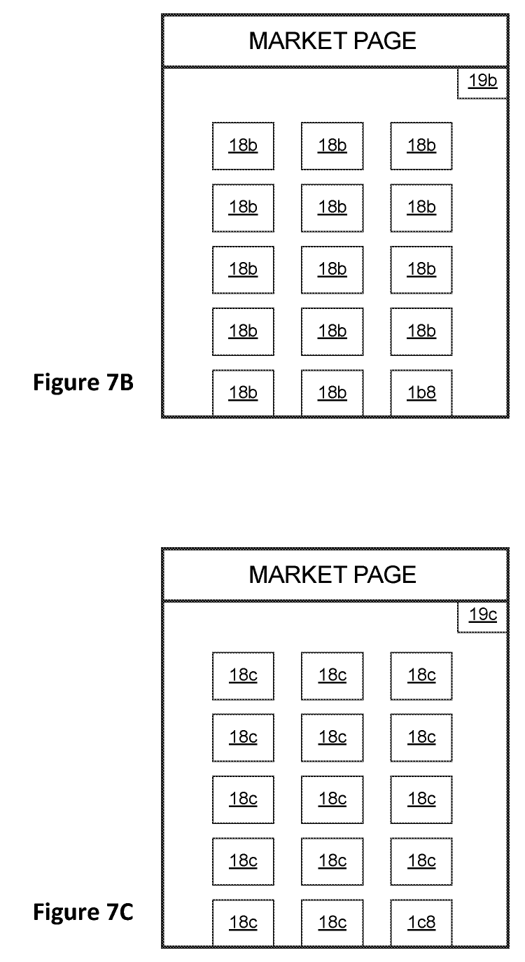

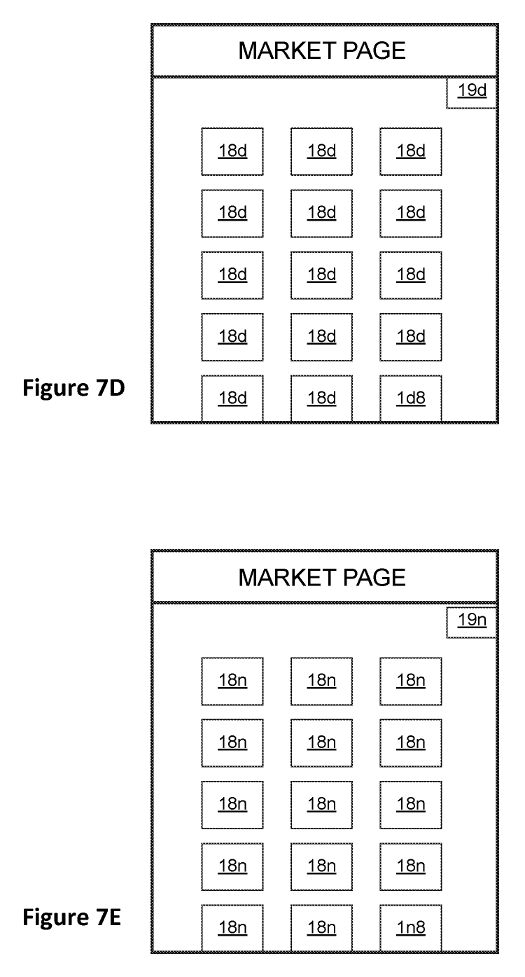

The present technology also provides user data personalization for data-driven marketable health items. It therefore provides informal health social support for users within a social media platform, alongside a marketing space to rate and purchase health products. FIGS. 7, 7A, 7B, 7C, 7D and 7E show non-limiting, exemplary user interfaces provided by an example social media multi-modal health technology platform—Market Pages. The Market Pages are pages for displaying advertisements and products that are harmonious to any of the categories of health on the identity page, and thus of some relevance to be additive to promoting health for the user's motivational influence and potential purchasing. The market pages include the potential for buying and selling of items. In some embodiments, this is a separate space and a mere online service for purchasing goods and services (with potential outsourcing with Pay Pal, Amazon/Amazon Prime, though not bound to this use).

Items 19 a-19e of FIG. 7 relate to an example of the visual representation of the digitally marketable health items via data-management and data-driven mechanisms, and allow for user personalization of the Market page via either hearting, liking, laughing, disliking, or a personalized option via clicking icons (represented conceptually as 19a-19d, . . . , 19n). To note, the number of user personalization options are not bound to this disclosure, and there could be n personalization options, where n is an infinite number of ways to personalize the Market page via these self-privatizing digital-visual and data-driven systems, to leave the design open for further reconstruction and freedom for option availability adjustments, as needed.

Example elements of the Market Pages are described below:

Item(s) 18 may be placeholders (shape of squares, as depicted, is not bound to this disclosure and thus provides simply a visual example) for such advertisements. Items 19a, 19b, 19c, 19d, and 19n in FIG. 7 relate to arrangement of displayed advertisements. These arrangements have been proposed to allow for personalization of the Market page, via either hearting, liking, laughing, disliking, or an unpersonalized option via clicking on icons (e.g., represented conceptually as 19a-19n), to allow for user personalization of the page. To note, the number of personalization options are also not bound in this disclosure, and thus there could be n personalization options, where n is an infinite number of ways to personalize, to leave the design open for further reconstruction and free adjustment. Moving forward with the description, these personalization options have been proposed to be adjusted on the page itself via emojis, and/or another small icon(s), or via the settings section on the profile page. Furthermore, specific details of the Market Page's placeholder(s) item(s) 18 may, for example, include 3D box or sphere zoom and rotation capabilities of the selected product, for user friendliness, and visual guiding of product information and exposure to the user/viewer.

FIGS. 7, 7A, 7B, 7C, 7D and 7E illustrate examples of ability for self-privatizing digital-visual and data-driven systems of user's data personalization for digitally marketable health items. In FIG. 7A, Item 18a displays all the items that were personalized with the Item 19a icon (i.e. if the icon is the hearting page for instance, though this detail is not bound within the details of discloser as previously mentioned). In FIG. 7B, Item 18b displays all the items that were personalized with the Item 19b icon (i.e. if the icon is the liking page for instance, though this detail is not bound within the details of discloser as previously mentioned). In FIG. 7C, Item 18c displays all the items that were personalized with the Item 19c icon (i.e. if the icon is the laughing page for instance, though this detail is not bound within the details of disclosure as previously mentioned). In FIG. 7D, Item 18d displays all the items that were personalized with the Item 19d icon (i.e. if the icon is the disliking page for instance, though this detail is not bound within the details of discloser). And so on, for other 19e-19n items, in which as mentioned before and shown in FIG. 7E, n is an infinite number of ways to personalize the market page in these particular layout tabs as drawn and exemplified, and are open for design reconstruction and free adjustment.

FIGS. 8A-8D depict non-limiting, exemplary user interfaces provided by an example social media multi-modal health technology platform—WHY Pages and some example variations in showing versing hiding subcategories of data information. In certain example embodiments, the WHY Pages may include each of the categories (aka modalities or determinants) of health (depicted in this discussed figure, “Body,” “Mind,” “Spirit,” to etc.) with bullet points (depicted as Item 20a, 20b, 20c, 20d, 20e, 20f, 20g, 20h, 20i, to etc.) that can be shown or hidden via selected clicks, for Why, What, and How. Within the Why, What, and How tabs, via shown versus hidden selected clicks, there will be further expansion of an outline of more options for data entries by the user. For instance, by clicking on the Body tab (depicted by the placeholder of 20a), it will show the Why, What, and How data text expansion and/or tabs respectively next to it and/or underneath it. Following clicking on 20a, for instance, it will say “Why is my Body important to me?” (to note: this phrasing, and for example, the word “important” shown in brackets in the figure, is not bound to the disclosure). There will then be a space and/or placeholder underneath to type in the user's responses (proposed examples will be shown in light grey ink or other colored ink; font color options are not bound to these figures' chosen uniform black ink). Examples of these spaces and/or placeholders are shown for instance in FIG. 8B, stating “ex) Because without it I can't function” and “ex) Because it is where my spirit resides.” There will also be an option to “(+) Add another reason,” for more personal data entries under the respectively bulleted category. FIGS. 8C and 8D display the same similarly structured format with specific examples listed as well, but for the WHAT bullet point (depicted as Item 20b) and the HOW bullet point (depicted as Item 20c) respectively. To note: on the HOW subcategories, there is also a listed bullet shown here as a heart emoji (which is not a detail that is bound in the disclosure, and thus the design for this is open to be freely changed, and is just an example) with text, shown as “<3 Click the heart icon for more support.” To note, the WHY, WHAT, and HOW bullet points and it's further tabbed and extended data forms thereafter, as aforementioned, will be similarly structured in each overarching category (or determinant or modality; depicted as Items 10a-10i, and etc. in previously described FIG. 4 as disclosed above, that the number of such categories is numerically unbound) of health for the WHY page.

One example of the present technology allows transferring health modalities and determinants (defined by topical bodies or integral components respectively for health foundations, i.e. nutrition, exercise, hydration, sleep, stress management, spirituality, etc.) into the numerical and visual representations. Users of a social media multi-modal health technology platform may enter or update information related to their health modalities and determinants via the system. FIGS. 9A-9B depict the two non-limiting, exemplary user interfaces provided by an example social media multi-modal health technology platform—Entry Pages. This is viewed by selecting one of the categories of health, shown on the Identity Page (i.e. Body, or for example Item 10f), which then takes the user to the first entry page, depicted in FIG. 9A. For instance, the top of FIG. 9A shows the first entry stating, “How would you currently rate yourself in this sphere of health?” And then below will be the circular entry rating system, that rates numbers on a 1-10 scale, with 1 being the lowest/least healthy, and 10 being the highest/healthiest. It has been proposed that the numerical entry can be conducted and set by moving the point of reference (a bar or a dot, for instance), clockwise to increase the number, and counterclockwise to decrease the number, starting from the top of the circle. In an example embodiment, the numerical data bar marker may be changed to be in a clockwise direction, with the 1 starting a little further from the 12 o'clock position if the sphere were a clock. The disclosure does not bind these detail specs to its design, but rather to the utility and function behind the platform.

FIGS. 9A-9B depict an example design of two rectangular shaped tick marks, with the starting tick mark (set at the top and immovable, as this is the starting point for inputting the rating quantity) having horizontal lines to fill it (for identification purposes of this disclosure). The other tick mark is similarly depicted as an example as a rectangle, but for figure item discernment purposes is depicted with vertical lines to fill the rectangular tick mark, and depicts the movable tick mark that will ultimately assign numerical value for the rating system, according to its placement. It has been proposed that a 5 numerical value for the rating system will have the vertical-line marked tick bar at the 6 o'clock position if the circle had the face of a clock, as shown in FIG. 9A. On the other hand, it has been proposed that a numerical value for the rating system of a 9 will have the vertical-line marked tick bar at about the 10:30 o'clock position if the circle had the face of a clock, as shown in FIG. 9B. The number 9 in FIG. 9B represents a 9 rating on a 1-10 scale (as an example), it does not represent the item 9 disclosed above.

Once the numerical entry has been set on an entry page, for instance in FIG. 9A, the user can then click “save entry” to move to the next entry page, (“save entry” is depicted in the bottom of FIG. 9A), or “cancel” to not save the entry and go back to the Identity Page (this canceling ability is not visibly marked/shown as a design feature in FIGS. 9A-9B, but has been proposed to be an “X” or another icon, text, etc. to indicate its appropriate function as stated here). If the entry is saved on the first entry page, then the second entry page will appear and depict the next question, for instance, “Where would you like to be rated in this sphere of health?”, as depicted in FIG. 9B. Similar to the former entry page's description, the numerical entry will be set on a 1-10 scale, and then after the number value is chosen with the movable tick mark placement (vertical lines drawn for discernment in rectangle tick mark, as depicted in FIGS. 9A-9B, for example), the user can click either “save entry” to complete the entry submission, or can hit “back” or “cancel” (not shown in FIGS. 9A-9B) to not save that entry, and return to the first entry page or the Identity Page respectively. If the entry is fully submitted (meaning both the first entry page and the second entry page have subsequently saved entries), then the entry will be displayed on the Identity page, either by itself or its average of the day's entries, or month's entries, etc. (it has been proposed, in which this average of entries over a time period, would be adjusted in settings for the user's preference of a displayed time period average on the Identity Page).

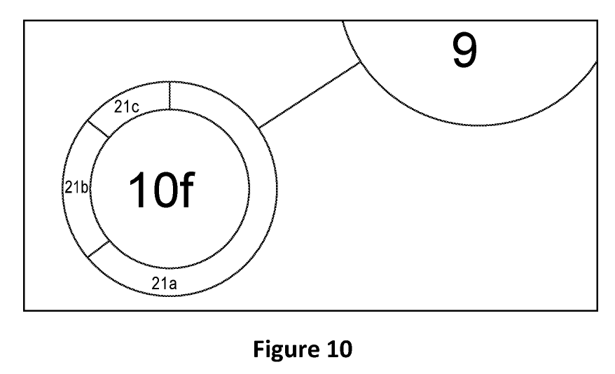

Example self-rating digital-visual systems according to the present technology disclosed herein can be split into two subsets. The first subset of self-rating digital-visual systems comprises the digital-visual representation for numerical data entry averages of where one is currently at with their health for a particular health modality or determinant (e.g., as depicted in item 21a in FIG. 10). The second subset of the self-rating digital-visual systems comprises the digital-visual representation for numerical data entry averages of where one would like to be at (health vision-orientation) with their health for a particular health modality or determinant (e.g., as depicted as 21b in FIG. 10). In an embodiment, this latter subset effectively is a digital average of a user's digital-visual information and representation of health vision for that particular modality or determinant of health.

As discussed above in connection with FIGS. 9A-9B, the self-rating digital-visual systems of health modalities enables user to input where they self-rate at present (e.g., Item 21a in FIG. 10) versus towards health vision (e.g., Item 21b in FIG. 10).

FIG. 10 shows a non-limiting, exemplary user interface provided by an example social media multi-modal health technology platform—an entry average of entry pages. The entry average will show up on the identity page after each entry (2 entries, exemplified in FIGS. 9A-9B, are required to complete a full entry submission) has been fully submitted, according to the set time frame for entry average viewing (i.e. total, a year, month, or day). In FIG. 10, following at least 1 fully submitted entry, the entry will show the first entry page submission rating or average of first entry page submitted entries per adjusted time frame, depicted in FIG. 10 as Item 21a, in juxtaposition with the second entry page submission rating or average of second entry page submitted entries per adjusted time frame, as depicted in FIG. 10 as Item 21b. The “9” in FIG. 10 represents the Item 9 displayed from the Identity page. To note: For an entry page's submission rating to be seen on the Identity page, it must be in the context of a fully submitted entry (meaning both the first entry and second entry pages have to both be submitted in succession to each other for either page to be taken into account in the average of entries displayed on the Identity Page's 21a and 21b depictions). Item 21c in FIG. 10 marks the space where there is nothing digitally-visually inputted by the user. For example, it may be a blank and empty space. The color to identify this space could be any color(s) and is not bound within the details of this disclosure.

The present technology therefore includes social support through a social-media platform, and a self-assessment rating system of a user's health modalities, to help the user move towards their health modality visions, while being socially supported along the way.

In certain example embodiments, colors for the Identity page's average numerical entry submission ratings, e.g., depicted as 21a and 21b, have been proposed to be adjusted in Settings. For example, 21a could be depicted as white and 21b could be depicted as green, if adjusted as such in the application's or website's Settings. As a side but important note, on the Items 10a-10i or as described earlier, the numerically definite or infinite spheres of health (which are left open to be remodeled in design, and thus unbound to details of the disclosure). However, it is also open to potentially have this be set to a max or strictly structured, in order to maintain good management of the Identity Page from a logistical standpoint. Moreover, it has been proposed to keep the spheres of health the same color, i.e. standard and unchangeable per user, for later functional use within the platform (for instance, showing who is active in which category of health in a color-coordinated and socially uniform manner, on the “My Fans” page, or “My Squad” page). However, this too is not a detail that is bound to the disclosure and thus is open to be remodeled and changed.

In terms of swiping direction dynamics for navigation through this social media platform, potential features and/or directions for user mobility within the application or website are described herein. In certain example embodiments, a website will have to be maneuvered differently than swiping due to the ubiquity of non-touch screen computers; for instance, arrows can be inserted on each page to click on the website version of this social media platform for directional navigation through the main pages. Thus, the note on swiping direction dynamics proposed is as followed: If a user is locationally situated at the Profile Page, and swipes from right to left (aka to the right), it takes the user to the Identity Page. From there, swiping from top to bottom (aka swiping down), takes the user to the Planet Page. Alternatively, swiping down to up (aka swiping up) from the Identity Page, will take the user to the Market Page. To note, from the Planet Page, swiping bottom to top (aka swiping up) will take the user back to the Identity Page. Also, if the user swipe from left to right (aka to the right), from the Identity Page, it will take the user back to the Profile Page. Swiping from right to left (aka to the left) from either the Planet Page, the Identity Page, or the Market Page, takes the user to the Social Page. In some example embodiments, a home or profile button (or visual icon) has also been proposed to be displayed on each page, to take the user back to the Profile Page, for user feasibility.

While the technology has been described in connection with what is presently considered to be the most practical and preferred embodiments, it is to be understood that the technology is not to be limited to the disclosed embodiments, but on the contrary, is intended to cover various modifications and equivalent arrangements included within the spirit and scope of the appended claims.

We claim:

1. A multi-modal health and social media system comprising a processing system including at least one processor, the processing system being configured to:
generate a data entry user interface enabling a user to enter information related to at least one modality of health, the entered information including a current value of the at least one modality of health accessed by the user and a desired value of the at least one modality of health from the user;
convert the entered information related to the at least one modality of health into converted information related to the at least one modality of health;
represent the converted information related to the at least one modality of health on a modality review user interface;
generate a post entry user interface enabling the user to submit one or more posts to a social media platform; and
display the posts submitted by the user as corresponding data entries on a planet respectively.

2. The multi-modal health and social media system according to claim 1, wherein the at least one modality of health comprises body, mind, soul, air, water, nutrition, exercise, community, sleep, shelter and/or love.

3. The multi-modal health and social media system according to claim 1, wherein the converted information includes information generated based on the current value of the at least one modality of health, and/or the desired value of the at least one modality of health.

4. The multi-modal health and social media system according to claim 1, wherein the converted information includes:
an average current value of the at least one modality of health averaged from the current value and other current values of the at least one modality of health previously entered by the user during a time period, and
an average desired value of the at least one modality of health averaged from the desired value and other desired values of the at least one modality of health previously entered by the user during the time period.

5. The multi-modal health and social media system according to claim 1, wherein the submitted one or more posts are added to the planet as pixilated formations.

6. The multi-modal health and social media system according to claim 1, wherein the submitted one or more posts comprises textual information, image(s), audio(s), and/or video(s).

7. The multi-modal health and social media system according to claim 1, wherein the processing system is further configured to:
detect a user input corresponding to a directional swipe; and
upon detecting the user input, cause the planet to rotate towards a direction indicated by the directional swipe.

8. The multi-modal health and social media system according to claim 1, wherein the processing system is further configured to cause the planet to zoom in or zoom out in accordance with a user input.

9. The multi-modal health and social media system according to claim 1, wherein the processing system is further configured to display details of a post upon detecting a data entry corresponding to the post is selected by a user.

10. The multi-modal health and social media system according to claim 9, wherein the processing system is further configured to generate a user interface enabling the user to edit the details of the post.

11. A non-transitory computer-readable storage medium having stored therein an information processing program for execution by a device comprising a processor operatively coupled to a display, the information processing program comprising instructions that, when executed by the processor, causing the device to perform operations including:
generating a data entry user interface enabling a user to enter information related to at least one modality of health, the entered information including a current value of the at least one modality of health accessed by the user and a desired value of the at least one modality of health from the user;
converting the entered information related to the at least one modality of health into converted information related to the at least one modality of health;
representing the converted information related to the at least one modality of health on a modality review user interface;
generating a post entry user interface enabling the user to submit one or more posts to a social media platform; and
displaying the posts submitted by the user as corresponding data entries on a planet respectively.

12. The non-transitory computer-readable storage medium claim 11, wherein the at least one modality of health comprises body, mind, soul, air, water, nutrition, exercise, community, sleep, shelter and/or love.

13. The non-transitory computer-readable storage medium claim 11, wherein converting the entered information related to the at least one modality of health into the converted information related to the at least one modality of health comprises:
generating an average current value of the at least one modality of health by averaging the current value and other current values of the at least one modality of health previously entered by the user during a time period, and
generating an average desired value of the at least one modality of health by averaging the desired value and other desired values of the at least one modality of health previously entered by the user during the time period.

14. The non-transitory computer-readable storage medium claim 11, wherein the submitted one or more posts are added to the planet as pixilated formations.

15. The non-transitory computer-readable storage medium claim 11, wherein the information processing program further comprises instructions that, when executed by the processor, causing the device to perform operations including:
detecting a user input corresponding to a directional swipe; and
upon detecting the user input, causing the planet to rotate towards a direction indicated by the directional swipe.

16. The non-transitory computer-readable storage medium claim 11, wherein the information processing program further comprises instructions that, when executed by the processor, causing the device to cause the planet to zoom in or zoom out in accordance with a user input.

17. A method for managing and sharing multi-modal health information, comprising:
generating a data entry user interface enabling a user to enter information related to at least one modality of health, the entered information including a current value of the at least one modality of health accessed by the user and a desired value of the at least one modality of health from the user;
converting the entered information related to the at least one modality of health into converted information related to the at least one modality of health;
representing the converted information related to the at least one modality of health on a modality review user interface;
generating a post entry user interface enabling the user to submit one or more posts to a social media platform; and
displaying the posts submitted by the user as corresponding data entries on a planet respectively.

18. The method according to claim 17, wherein the at least one modality of health comprises body, mind, soul, air, water, nutrition, exercise, community, sleep, shelter and/or love.

19. The method according to claim 17, wherein the submitted one or more posts are added to the planet as pixilated formations.

20. The method according to claim 17, further comprising:
detecting a user input corresponding to a directional swipe;
upon detecting the user input, causing the planet to rotate towards a direction indicated by the directional swipe.
